(19) United States
(12) Patent Application Publication
(10) Pub. No.: US 2024/0217940 A1
(43) Pub. Date: Jul. 4, 2024

(54) PROCESSES FOR PRODUCING BIOMONOMERS AND PRECURSORS FOR THE BIOMONOMERS

(71) Applicant: UOP LLC, Des Plaines, IL (US)

(72) Inventors: Yili Shi, Buffalo Grove, IL (US); Veronica G. Deak, Chicago, IL (US); Kristen E. Allaire, Chicago, IL (US); Joel T. Walenga, Lake Zurich, IL (US)

(21) Appl. No.: 18/506,347

(22) Filed: Nov. 10, 2023

Related U.S. Application Data
(60) Provisional application No. 63/477,857, filed on Dec. 30, 2022.

(51) Int. Cl. C07D 307/68

(52) U.S. Cl. CPC C07D 307/68

(57) ABSTRACT

Processes for producing biomonomers and precursors for same with a slurry-phase reaction. A furoate carboxylation reaction is conducted within a hydrocarbon slurry which includes carbon dioxide. The reaction produces dicarboxylates which can be separated from the slurry and used to produce biomonomers like furan dicarboxylate methyl ester and furan dicarboxylic acid.

### RELATED APPLICATIONS

This application claims priority to U.S. Provisional Patent Application Ser. No. 63/477,857, filed Dec. 30, 2022, the entirety of which is incorporated herein by reference.

### FIELD OF THE INVENTION

This invention relates generally to process for the production of aromatic carboxylic acid compounds including furan dicarboxylic acid and furan dicarboxylate methyl ester, and precursors for same, from biomass.

### BACKGROUND OF THE INVENTION

Recently, processes have been developed for producing aromatic carboxylic acids and esters from sugars produced from biomass. These aromatic carboxylic acids and esters can be converted to dicarboxylates which can in turn be utilized to produce monomers like furan dicarboxylate methyl ester (FDME) and furan dicarboxylic acid (FDCA). As is known, these monomers are useful in making polymers and plastics and, since they are at least in part derived from biomass, may be referred to as biomonomers.

These processes are desirable because they provide for the production of the biomonomers as opposed to producing chemicals and monomers from fossil fuel sources.

Additionally, the processes are desirable because they may consume carbon dioxide—which is considered a greenhouse gas.

While generally effective for their intended purposes, these carboxylation reactions are conducted as solid-state melt reactions, which severely complicates the design of the necessary reactor.

Accordingly, it would be desirable to provide processes which produce biomonomers from biomass derived components and carbon dioxide which do not require complex reactors.

### SUMMARY OF THE INVENTION

The present inventors have invented processes for producing biomonomers and precursors for same which utilize a slurry-phase reaction. In particular, the present invention addresses the shortcomings of conventional processes by conducting a furoate carboxylation reaction within a hydrocarbon slurry containing the furoate, an alkali base, and carbon dioxide. A slurry-phase carboxylation reaction/process is advantageous because it allows for the utilization of a slurry-bubble column reactor design. Additionally, such a reaction allows for the necessary reaction heat may be added via the hydrocarbon oil. Furthermore, heat may be recovered from spent hydrocarbon oil via heat exchange. Similarly, exothermic heat that would cause selectivity loss may be removed. Finally, the reactor may have a counter-current oil/gas flow design to favor reactions.

Therefore, the present invention may be characterized, in at least one aspect, as providing a process for producing precursors for producing bio-monomers by: forming a slurry comprising furoates and an alkali base; and, heating the slurry in the presence of carbon dioxide to form dicarboxylates.

The slurry may further include a carboxylate reaction promoter.

The slurry may be heated to a temperature between 150° C. to 360° C. at a pressure up to 6,895 kPa (1,000 psi).

The carbon dioxide may be provided as bubbles, and the bubbles may flow counter current to the slurry.

The slurry may be formed in a hydrocarbon having negligible solubility to the furoates and the alkali base.

The process may also include recovering the dicarboxylates and converting the dicarboxylates to furan dicarboxylate methyl ester or furan dicarboxylic acid, or both.

The alkali base, a furoate counter ion, or both may be selected from a group consisting of: lithium, sodium, potassium, rubidium, cesium, and mixtures thereof.

The slurry may further include cesium.

The present invention may also be characterized broadly, in at least one aspect, as providing a process for producing furan dicarboxylate methyl ester or furan dicarboxylic acid from a biomass derived compound by: passing a slurry comprising furoates and an alkali base in a vessel in a reaction zone; passing carbon dioxide into the vessel to contact the slurry; heating the slurry to form dicarboxylates; recovering the dicarboxylates; and, converting the dicarboxylates to furan dicarboxylate methyl ester or furan dicarboxylic acid, or both.

The slurry may also include comprises a carboxylate reaction promoter.

The alkali base, or a furoate counterion, or both may be selected from a group consisting of: lithium, sodium, potassium, rubidium, cesium, and mixtures thereof.

The slurry may be heated to a temperature between 150° C. to 360° C., at a pressure up to 6,895 kPa (1,000 psi).

The carbon dioxide may be passed into the vessel in bubbles which flow counter current to the slurry.

The slurry may be formed in a hydrocarbon having negligible solubility to the furoates and the alkali base. The dicarboxylates may be recovered by separating the hydrocarbon from the dicarboxylates. The process may include recycling the separated hydrocarbon for forming the slurry. The process also may include recovering heat from the separated hydrocarbon in a heat exchanger.

The furan dicarboxylate methyl ester may be dimethyl furan-2,5-dicarboxylate and the furan dicarboxylic acid may be furan-2,5-dicarboxylate.

The slurry may further include cesium.

Additional aspects, embodiments, and details of the invention, all of which may be combinable in any manner, are set forth in the following detailed description of the invention.

### DETAILED DESCRIPTION OF THE INVENTION

As mentioned above, the present invention provides processes which use a slurry phase carboxylation reaction for furoates. Producing furoates from biomass is known. See, U.S. Pat. Nos. 7,572,925 and 8,772,515. As used herein “biomass” includes, but is not limited to, lignin, plant parts, fruits, vegetables, plant processing waste, wood chips, chaff, grain, grasses, corn, corn husks, weeds, aquatic plants, hay, paper, paper products, recycled paper and paper products, and any cellulose, lignin, or combinations thereof containing biological material or material of biological origin.

Accordingly, the process is intended to be used as part of an integrated C5 biomass to FDCA/FDME production facility; however, other implementations may be utilized.

In general, the process mixes furoates such as furoic acid salts, an alkali base, and any promoters in a hydrocarbon oil to form a slurry. This slurry is heated to reaction temperature in the presence of a carbon dioxide gas, potentially in a counter-current slurry-bubble column reactor. The furoates are converted to dicarboxylates (FDCA salts) and are then recovered from the oil slurry. The dicarboxylates can then be converted, as is known, in subsequent chemicals steps into either FDCA free-acid or FDME.

With these general principles in mind, one or more embodiments of the present invention will be described with the understanding that the following description is not intended to be limiting.

Methods according to the present invention include forming a slurry comprising furoates and an alkali base. A furoate counter ion may include lithium, sodium, potassium, rubidium, cesium, and mixtures thereof.

The alkali base may be a metal hydroxide such as lithium hydroxide, sodium hydroxide, potassium hydroxide, rubidium hydroxide, cesium hydroxide, magnesium hydroxide, calcium hydroxide, and mixtures thereof.

The alkali base may be at a mole ratio of alkali base to furoate may be from about 1:1 to 2:1, 1:0.1 to 1:1, 1:0.1 to 1:0.5 or 0.1:1 to 1:1.

The slurry may be formed in a hydrocarbon oil a hydrocarbon material containing between 5 to 30 carbon atoms per molecule and having paraffinic and/or aromatic functional groups. In general, the hydrocarbon oil selected for the slurry will have negligible solubility to the furoate and the alkali base.

The slurry may further include a carboxylate reaction promoter, such as a hydrocarbon with an alpha C-H bond, like acetate. For example, acetates may be selected from propionate, butyrate, isobutyrate and lactate.

Carbon dioxide is provided to the slurry. For example, the carbon dioxide can be provided as bubbles into the slurry. The bubbles may flow counter current to the flow of the slurry.

With the carbon dioxide, the slurry is heated to a temperature of between 150 to 360° C., or between 270 to 330° C., at a pressure from about atmospheric up to 6,895 kPa (1,000 psi), or up to 4,826 kPa (700 psig), or up to 4,137 kPa (600 psig) and sufficient heat for a time sufficient to form dicarboxylates via a carboxylation reaction between the carbon dioxide and the furoate. The reaction time is sufficient to produce the aromatic carboxylic acid compound is from about 1 second to 24 hours, 1 minute to 12 hours, 1 minute to 6 hours, or 1 minute to 1 hour. The process may be continuous, semi-batch or batch reaction process.

The dicarboxylates that are made can include terephthalic acid, naphthalic acid, thiophene dicarboxylic acid, pyridine dicarboxylic acid, carbazole dicarboxylic acid, and dibenzothiophene dicarboxylic acid. In particular, the dicarboxylates may be furan dicarboxylate, and specifically, furan-2,-dicarboxylate and/or furan-2,5- dicarboxylate.

The dicarboxylates may be recovered by being separated from the slurry. The recovered decarboxylates may be converted to FDME, FDCA, or both. In particular, the produced biomonomers may include one or more of furan-2,5-dicarboxylic acid, furan 2,4 dicarboxylic acid, dimethyl furan-2,5-dicarboxylate, dimethyl furan-2,4-dicarboxylate, and salts thereof. These biomonomers may converted in polymers as is known in the art.

After the decarboxylates have been separated, the separated slurry may be recycled. Additionally, heat may be recovered from the separated slurry in a heat exchanger.

Compared with existing reactors and processes the slurry reactor and reaction process is easier to implement and provides an effective and efficient means for producing biomonomers and components therefor.

### EXPERIMENTS

Three different slurries were formed based on the components and in the ratio specified below in TABLE 1.

TABLE 1

| | Example 1 | Example 2 | Example 3 |
|---|---|---|---|
| 1 molar equivalent M- | K-Furoate | K-Furoate | Cs-Furoate |
| Furoate | | | |
| 0.55 molar equivalent | K₂CO₃ | K₂CO₃ | Cs₂CO₃ |
| M₂—CO₃ | | | |
| 0.35 molar equivalent | K-Acetate | K-Acetate | none |
| M-Acetate | | | |

Examples 1, 2, and 3, were heated, in the presence of carbon dioxide, for 5 hours at a temperature of 315° C., 325° C., and 250° C.

The furoate conversion and FDCA yield for the Examples are shown below in TABLE 2.

TABLE 2

| | Example 1 | Example 2 | Example 3 |
|---|---|---|---|
| M-furoate conversion, | 55 | 96 | 99 |
| mol % | | | |
| M₂-FDCA yield, wt % | 33 | 54 | 84 |

### SPECIFIC EMBODIMENTS

While the following is described in conjunction with specific embodiments, it will be understood that this description is intended to illustrate and not limit the scope of the preceding description and the appended claims.

A first embodiment of the invention is a process for producing precursors for producing bio-monomers, the process comprising forming a slurry comprising furoates and an alkali base; and, heating the slurry in the presence of carbon dioxide to form dicarboxylates. An embodiment of the invention is one, any or all of prior embodiments in this paragraph up through the first embodiment in this paragraph, wherein the slurry further comprises a carboxylate reaction promoter. An embodiment of the invention is one, any or all of prior embodiments in this paragraph up through the first embodiment in this paragraph, wherein the slurry is heated to a temperature between 150° C. to 360° C. at a pressure up to 6,895 kPa (1,000 psi). An embodiment of the invention is one, any or all of prior embodiments in this paragraph up through the first embodiment in this paragraph, wherein the carbon dioxide is provided in bubbles. An embodiment of the invention is one, any or all of prior embodiments in this paragraph up through the first embodiment in this paragraph, wherein the bubbles flow counter current to the slurry. An embodiment of the invention is one, any or all of prior embodiments in this paragraph up through the first embodiment in this paragraph, wherein the slurry is formed in a hydrocarbon having negligible solubility to the furoates and the alkali base. An embodiment of the invention is one, any or all of prior embodiments in this paragraph up through the first embodiment in this paragraph, further comprising recovering the dicarboxylates; and, converting the dicarboxylates to furan dicarboxylate methyl ester or furan dicarboxylic acid, or both. An embodiment of the invention is one, any or all of prior embodiments in this paragraph up through the first embodiment in this paragraph, wherein the alkali base, a furoate counter ion, or both are selected from a group consisting of lithium, sodium, potassium, rubidium, cesium, and mixtures thereof. An embodiment of the invention is one, any or all of prior embodiments in this paragraph up through the first embodiment in this paragraph, wherein the slurry further comprises cesium.

A second embodiment of the invention is a process for producing furan dicarboxylate methyl ester or furan dicarboxylic acid from a biomass derived compound, the process comprising passing a slurry comprising furoates and an alkali base in a vessel in a reaction zone; passing carbon dioxide into the vessel to contact the slurry; heating the slurry to form dicarboxylates; and, recovering the dicarboxylates; and, converting the dicarboxylates to furan dicarboxylate methyl ester or furan dicarboxylic acid, or both. An embodiment of the invention is one, any or all of prior embodiments in this paragraph up through the second embodiment in this paragraph, wherein the slurry further comprises a carboxylate reaction promoter. An embodiment of the invention is one, any or all of prior embodiments in this paragraph up through the second embodiment in this paragraph, wherein the alkali base, or a furoate counterion, or both are selected from a group consisting of lithium, sodium, potassium, rubidium, cesium, and mixtures thereof. An embodiment of the invention is one, any or all of prior embodiments in this paragraph up through the second embodiment in this paragraph, wherein the slurry is heated to a temperature between 150° C. to 360° C., at a pressure up to 6,895 kPa (1,000 psi). An embodiment of the invention is one, any or all of prior embodiments in this paragraph up through the second embodiment in this paragraph, wherein the carbon dioxide is passed into the vessel in bubbles which flow counter current to the slurry. An embodiment of the invention is one, any or all of prior embodiments in this paragraph up through the second embodiment in this paragraph, wherein the slurry is formed in a hydrocarbon having negligible solubility to the furoates and the alkali base. An embodiment of the invention is one, any or all of prior embodiments in this paragraph up through the second embodiment in this paragraph, wherein recovering the dicarboxylates comprises separating the hydrocarbon from the dicarboxylates. An embodiment of the invention is one, any or all of prior embodiments in this paragraph up through the second embodiment in this paragraph, further comprising recycling the separated hydrocarbon for forming the slurry. An embodiment of the invention is one, any or all of prior embodiments in this paragraph up through the second embodiment in this paragraph, further comprising recovering heat from the separated hydrocarbon in a heat exchanger. An embodiment of the invention is one, any or all of prior embodiments in this paragraph up through the second embodiment in this paragraph, wherein the slurry further comprises cesium. An embodiment of the invention is one, any or all of prior embodiments in this paragraph up through the second embodiment in this paragraph, wherein the furan dicarboxylate methyl ester comprises dimethyl furan-2,5-dicarboxylate and, wherein the furan dicarboxylic acid comprises furan-2,5-dicarboxylate.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for producing precursors for producing bio-monomers, the process comprising:
forming a slurry comprising furoates and an alkali base; and, heating the slurry in the presence of carbon dioxide to form dicarboxylates.

2. The process of claim 1, wherein the slurry further comprises a carboxylate reaction promoter.

3. The process of claim 1, wherein the slurry is heated to a temperature between 150° C. to 360° C. at a pressure up to 6,895 kPa (1,000 psi).

4. The process of claim 1, wherein the carbon dioxide is provided in bubbles.

5. The process of claim 3, wherein the bubbles flow counter current to the slurry.

6. The process of claim 1, wherein the slurry is formed in a hydrocarbon having negligible solubility to the furoates and the alkali base.

7. The process of claim 1, further comprising:
recovering the dicarboxylates; and,
converting the dicarboxylates to furan dicarboxylate methyl ester or furan dicarboxylic acid, or both.

8. The process of claim 1, wherein the alkali base, a furoate counter ion, or both are selected from a group consisting of: lithium, sodium, potassium, rubidium, cesium, and mixtures thereof.

9. The process of claim 1, wherein the slurry further comprises cesium.

10. A process for producing furan dicarboxylate methyl ester or furan dicarboxylic acid from a biomass derived compound, the process comprising:
passing a slurry comprising furoates and an alkali base in a vessel in a reaction zone;
passing carbon dioxide into the vessel to contact the slurry;
heating the slurry to form dicarboxylates;
recovering the dicarboxylates; and,
converting the dicarboxylates to furan dicarboxylate methyl ester or furan dicarboxylic acid, or both.

11. The process of claim 10, wherein the slurry further comprises a carboxylate reaction promoter.

12. The process of claim 10, wherein the alkali base, or a furoate counterion, or both are selected from a group consisting of: lithium, sodium, potassium, rubidium, cesium, and mixtures thereof.

13. The process of claim 10, wherein the slurry is heated to a temperature between 150° C. to 360° C., at a pressure up to 6,895 kPa (1,000 psi).

14. The process of claim 10, wherein the carbon dioxide is passed into the vessel in bubbles which flow counter current to the slurry.

15. The process of claim 10, wherein the slurry is formed in a hydrocarbon having negligible solubility to the furoates and the alkali base.

16. The process of claim 15, wherein recovering the dicarboxylates comprises separating the hydrocarbon from the dicarboxylates.

17. The process of claim 16, further comprising:
recycling the separated hydrocarbon for forming the slurry.

18. The process of claim 16, further comprising:
recovering heat from the separated hydrocarbon in a heat exchanger.

19. The process of claim 10, wherein the slurry further comprises cesium.

20. The process of claim 10, wherein the furan dicarboxylate methyl ester comprises dimethyl furan-2,5-dicarboxylate and, wherein the furan dicarboxylic acid comprises furan-2,5-dicarboxylate.
